SEQUENCE LISTING (13 sequences)

SEQ ID NO: 1 - DNA, 12 bp, artificial sequence
  synthetic oligonucleotide
tcgagcgttgtc

SEQ ID NO: 2 - DNA, 20 bp, artificial sequence
  synthetic oligonucleotide
gggggacgatcgtcgggggg

SEQ ID NO: 3 - DNA, 20 bp, artificial sequence
  synthetic oligonucleotide
tccatgacgttcctgatgct

SEQ ID NO: 4 - DNA, 24 bp, artificial sequence
  synthetic oligonucleotide
tcgtcgttttgtcgttttgtcgtt

SEQ ID NO: 5 - DNA, 21 bp, artificial sequence
  synthetic oligonucleotide
tcgtcgaacgttcgagatgat

SEQ ID NO: 6 - DNA, 20 bp, artificial sequence
  synthetic oligonucleotide
tttttttttttttttttttt

SEQ ID NO: 7 - DNA, 20 bp, artificial sequence
  synthetic oligonucleotide
tttttttttttttttttttt

SEQ ID NO: 8 - DNA, 5 bp, artificial sequence
  synthetic oligonucleotide
ttttt

SEQ ID NO: 9 - DNA, 8 bp, artificial sequence
  synthetic oligonucleotide
tttttttt

SEQ ID NO: 10 - DNA, 11 bp, artificial sequence
  synthetic oligonucleotide
ttttttttttt

SEQ ID NO: 11 - DNA, 18 bp, artificial sequence
  synthetic oligonucleotide
tttttttttttttttttt

SEQ ID NO: 12 - DNA, 18 bp, artificial sequence
  synthetic oligonucleotide
aaaaaaaaaaaaaaaaaa

SEQ ID NO: 13 - DNA, 18 bp, artificial sequence
  synthetic oligonucleotide
cccccccccccccccccc

(19) United States
(12) Patent Application Publication
(10) Pub. No.: US 2011/0070575 A1
(43) Pub. Date: Mar. 24, 2011

(54) Immunomodulatory Compositions, Combinations and Methods

(75) Inventors: Keith B. Gorden, Woodbury, MN (US); Xiaohong Qui, Rosemount, MN (US); Paul D. Wightman, Woodbury, MN (US)

(73) Assignee: Coley Pharmaceutical Group, Inc., Wellesley, MA (US)

(21) Appl. No.: 11/720,862

(22) PCT Filed: Dec. 8, 2005
(86) PCT No.: PCT/US05/44306

§ 371 Date: Jun. 5, 2007

Related U.S. Application Data
(60) Provisional application No. 60/634,145, filed on Dec. 8, 2004.

(51) Int. Cl. A61K 31/70; C12N 5/06; A61P 37/00

(52) U.S. Cl. 435/6; 435/375

(57) ABSTRACT

The invention provides immunomodulatory compositions, immunomodulatory combinations, and methods of modulating TLR7-mediated biological activity. Generally, the immunomodulatory compositions include an immunomodulatory oligonucleotide in an amount effective to reduce TLR7-mediated biological activity. In some cases, an immunomodulatory combination can further include an IRM compound. In some of these embodiments, the IRM compound can be a TLR7/8 agonist. Generally, the Imethods include contacting immune cells with an immunomodulatory composition in an amount effective to reduce TLR7-mediated biological activity.

### BACKGROUND

There has been a major effort in recent years, with significant success, to discover new drug compounds that act by stimulating certain key aspects of the immune system, as well as by suppressing certain other aspects (see, e.g., U.S. Pat. Nos. 6,039,969 and 6,200,592). These compounds, referred to herein as immune response modifiers (IRMs), appear to act through basic immune system mechanisms known as Toll-like receptors (TLRs) to induce selected cytokine biosynthesis, induction of co-stimulatory molecules, and increased antigen-presenting capacity. They may be useful for treating a wide variety of diseases and conditions. For example, certain IRMs may be useful for treating viral diseases (e.g., human papilloma virus, hepatitis, herpes), neoplasias (e.g., basal cell carcinoma, squamous cell carcinoma, actinic keratosis, melanoma), and T_{H}2-mediated diseases (e.g., asthma, allergic rhinitis, atopic dermatitis), auto-immune diseases (e.g., multiple sclerosis), and are also useful as vaccine adjuvants.

Many of the IRM compounds are small organic molecule imidazoquinoline amine derivatives (see, e.g., U.S. Pat. No. 4,689,338), but a number of other compound classes are known as well (see, e.g., U.S. Pat. Nos. 5,446,153; 6,194,425; and 6,110,929) and more are still being discovered.

Certain small molecule IRMs (smIRMs) possess potent immunomodulating activity such as, for example, antiviral and antitumor activity. Certain smIRMs modulate the production and secretion of cytokines. For example, certain smIRM compounds induce the production and secretion of cytokines such as, e.g., Type I interferons, TNF-α, IL-1, IL-6, IL-8, IL-10, IL-12, MIP-1, and/or MCP-1. As another example, certain smIRM compounds can inhibit production and secretion of certain T_{H}2 cytokines, such as IL-4 and IL-5. Additionally, some smIRM compounds are said to suppress IL-1 and TNF (U.S. Pat. No. 6,518,265).

Other IRMs have higher molecular weights, such as, for example, oligonucleotides, including CpG oligodinucleotides (ODNs, see, e.g., U.S. Pat. No. 6,194,388). At least three structurally distinct classes of synthetic CpG ODNs have been described. CpG-B ODNs (also referred to as K-type CpG ODNs) can trigger the differentiation of antigen presenting cells (APCs) and the proliferation of B cells. CpG-A ODNs (also referred to as D-type CpG ODNs) can directly induce the secretion of interferon-α (IFN-α) from plasmacytoid dendritic cells (pDCs), which indirectly supports the subsequent maturation of APCs. CpG-C ODNs can stimulate B cells to secrete interleukin-6 (IL-6) and pDCs to produce IFN-α, thereby combining some of the stimulatory properties of CpG-A ODNs and CpG-B ODNs.

In view of the great therapeutic potential for IRMs, and despite the important work that has already been done, there is a substantial ongoing need to expand their uses and therapeutic benefits.

### SUMMARY

It has been found that certain oligonucleotide sequences, even some that previously have been identified as immunostimulatory, can reduce or even eliminate certain immunostimulatory activity of certain small molecule IRMs.

Accordingly, the present invention provides immunomodulatory compositions and methods of limiting TLR7-mediated biological activity of immune cells. Generally, the method includes contacting the immune cells with an immunomodulatory composition that includes an immunomodulatory oligonucleotide in an amount effective to reduce a TLR7-mediated biological activity of the cells. In some cases, the immunomodulatory oligonucleotide can include a CpG oligonucleotide.

In another aspect, the present invention also provides an immunomodulatory combination that includes a TLR7 agonist and an immunomodulatory oligonucleotide in an amount effective to reduce at least one TLR7-mediated biological activity induced by the TLR7 agonist. In some embodiments, the TLR7 agonist can be a small molecule IRM compound. In some embodiments, the immunomodulatory oligonucleotide can include a CpG oligonucleotide.

In yet another aspect, the present invention provides a method of selectively inhibiting TLR7-mediated biological activity of an IRM compound that is an agonist of TLR7 and at least one other TLR agonist. Generally, the method includes combining the IRM compound with an immunomodulatory oligonucleotide in an amount effective to reduce TLR7-mediated biological activity induced by the IRM compound; and contacting the combination of IRM compound and immunomodulatory oligonucleotide with immune cells capable of generating a TLR7-mediated biological response.

Various other features and advantages of the present invention should become readily apparent with reference to the following detailed description, examples, claims and appended drawings. In several places throughout the specification, guidance is provided through lists of examples. In each instance, the recited list serves only as a representative group and should not be interpreted as an exclusive list.

### BRIEF DESCRIPTION OF THE DRAWINGS

FIG. 1 shows inhibition of smIRM-induced TLR7-mediated biological activity by CpG ODN immunomodulatory oligonucleotides in a transfected cell line.

FIG. 2 shows inhibition of smIRM-induced TLR7-mediated biological activity by CpG ODN immunomodulatory oligonucleotides in a transfected cell line.

FIG. 3 shows inhibition of smIRM-induced TLR7-mediated biological activity by CpG ODN immunomodulatory oligonucleotides in peripheral blood mononuclear cells (PBMCs).

FIG. 4 shows inhibition of smIRM-induced TLR7-mediated biological activity by CpG ODN immunomodulatory oligonucleotides in peripheral blood mononuclear cells (PBMCs).

FIG. 5 shows inhibition of smIRM-induced TLR7-mediated biological activity by poly(T) immunomodulatory oligonucleotides in peripheral blood mononuclear cells (PBMCs).

FIG. 6 shows inhibition of smIRM-induced TLR7-mediated biological activity by poly(T) immunomodulatory oligonucleotides of varying lengths in a transfected cell line.

FIG. 7 shows inhibition of smIRM-induced TLR7-mediated biological activity by poly(T), poly(A), and poly(C) immunomodulatory oligonucleotides in a transfected cell line.

### DETAILED DESCRIPTION OF ILLUSTRATIVE EMBODIMENTS OF THE INVENTION

The present invention exploits the observation that certain oligonucleotide sequences can inhibit certain TLR7-mediated biological activities in a dose dependent manner. In one aspect, the invention provides a method of reducing TLR7-mediated biological activity of immune cells. In practice, the method may be used, for example, to limit undesirable effects experienced by a subject who has received a dose of a smIRM that is greater than necessary. As another example, the method may be used to decrease the activity of certain smIRMs that, alone, may induce too much TLR7-mediated biological activity to be clinically useful. In another aspect, the invention provides immunomodulatory combinations that include a TLR7 agonist and an immunomodulatory oligonucleotide in an amount effective to reduce TLR7-mediated biological activity induced by the TLR7 agonist.

For purposes of this invention, the following terms shall have the meanings set forth as follows:

“Agonist” refers to a compound that can combine with a receptor (e.g., a TLR) to induce a biological activity. An agonist may be a ligand that directly binds to the receptor. Alternatively, an agonist may combine with a receptor indirectly by, for example, (a) forming a complex with another molecule that directly binds to the receptor, or (b) otherwise results in the modification of another compound so that the other compound directly binds to the receptor (e.g., cellular signaling). An agonist may be referred to as an agonist of a particular TLR (e.g., a TLR7 agonist) or a particular combination of TLRs (e.g., a TLR 7/8 agonist—an agonist of both TLR7 and TLR8).

“Agonist-receptor interaction” refers to any direct or indirect interaction such as, for example, binding, forming a complex, or biochemical modification that induces a cellular activity.

“Immune cell” refers to cell of the immune system, i.e., a cell directly or indirectly involved in the generation or maintenance of an immune response, regardless of whether the immune response is innate or acquired, humoral or cell-mediated.

“Immunomodulatory oligonucleotide” refers to an oligonucleotide sequence that is capable of measurably inhibiting TLR7-mediated biological activity.

“Induce” and variations thereof refer to any measurable increase in biological activity. For example, induction of a particular cytokine refers to an increase in the production of the cytokine.

“Inhibit” and variations thereof refer to any measurable reduction of biological activity. For example, inhibition of a particular cytokine refers to a decrease in production of the cytokine. The extent of inhibition may be characterized as a percentage of a normal level of activity.

“IRM compound” refers generally to a compound that alters the level of one or more immune regulatory molecules, e.g., cytokines or co-stimulatory markers, when administered to an IRM-responsive cell. Representative IRM compounds include the small organic molecules, purine derivatives, small heterocyclic compounds, amide derivatives, and oligonucleotide sequences described below.

“Selective” and variations thereof refer to having a differential impact on biological activity to any degree. An agonist that selectively modulates biological activity through a particular TLR may be a TLR-selective agonist. TLR-selectivity may be described with respect to a particular TLR (e.g., TLR8-selective) or with respect to a particular combination of TLRs (e.g., TLR 7/9-selective). A TLR selective (e.g., TLR8-selective) compound may exclusively induce biological activity mediated by the indicated TLR (i.e., TLR-specific), or may induce biological activity mediated through multiple TLRs, but induce activity mediated through the indicated TLR to a greater extent than any other TLR (i.e., TLR-dominant such as, for example, TLR8-dominant).

“smIRM” refers generally to a small molecule IRM compound, an IRM compound having a molecular weight of about 1 kilodalton (kDa) or less.

“TLR-mediated” refers to a biological activity (e.g., cytokine production) that results, directly or indirectly, from TLR function. A particular biological activity may be referred to as mediated by a particular TLR (e.g., “TLR7-mediated”).

Also herein, the recitations of numerical ranges by endpoints include all numbers subsumed within that range (e.g., 1 to 5 includes 1, 1.5, 2, 2.75, 3, 3.80, 4, 5, etc.).

The TLR agonism for a particular compound may be assessed in any suitable manner. For example, assays and recombinant cell lines suitable for detecting TLR agonism of test compounds are described, for example, in U.S. Patent Publication Nos. US2004/0014779, US2004/0132079, US2004/0162309, US2004/0171086, US2004/0191833, and US2004/0197865.

Regardless of the particular assay employed, a compound can be identified as an agonist of a particular TLR if performing the assay with a compound results in at least a threshold increase of some biological activity mediated by the particular TLR. Conversely, a compound may be identified as not acting as an agonist of a specified TLR if, when used to perform an assay designed to detect biological activity mediated by the specified TLR, the compound fails to elicit a threshold increase in the biological activity. Unless otherwise indicated, an increase in biological activity refers to an increase in the same biological activity over that observed in an appropriate control. An assay may or may not be performed in conjunction with the appropriate control. With experience, one skilled in the art may develop sufficient familiarity with a particular assay (e.g., the range of values observed in an appropriate control under specific assay conditions) that performing a control may not always be necessary to determine the TLR agonism of a compound in a particular assay.

The precise threshold increase of TLR-mediated biological activity for determining whether a particular compound is or is not an agonist of a particular TLR in a given assay may vary according to factors known in the art including but not limited to the biological activity observed as the endpoint of the assay, the method used to measure or detect the endpoint of the assay, the signal-to-noise ratio of the assay, the precision of the assay, and whether the same assay is being used to determine the agonism of a compound for both TLRs. Accordingly it is not practical to set forth generally the threshold increase of TLR-mediated biological activity required to identify a compound as being an agonist or a non-agonist of a particular TLR for all possible assays. Those of ordinary skill in the art, however, can readily determine the appropriate threshold with due consideration of such factors.

Assays employing HEK293 cells transfected with an expressible TLR structural gene may use a threshold of, for example, at least a three-fold increase in a TLR-mediated biological activity (e.g., NFκB activation) when the compound is provided at a concentration of, for example, from about 1 μM to about 10 μM for identifying a compound as an agonist of the TLR transfected into the cell. However, different thresholds and/or different concentration ranges may be suitable in certain circumstances. Also, different thresholds may be appropriate for different assays.

In one aspect, the invention provides a method of limiting TLR7-mediated biological activity of immune cells. In practice, the method may be used, for example, to limit undesirable effects experienced by a subject who has received a dose of an IRM compound that is greater than necessary.

In other cases, for example, the method may be used to limit—or even eliminate—TLR7-mediated biological activity induced by a compound that is an agonist of TLR7 and at least one other TLR (e.g., TLR8 or TLR9). Thus, the method may be used to decrease TLR7-mediated biological activity so that the compound acts essentially as a dominant or even specific agonist of the other TLR. For example, reducing—or even eliminating—the TLR7-mediated biological activity of a TLR7/8 agonist may make the compound act essentially as a TLR8-selective agonist (e.g., as a TLR8-dominant agonist or a TLR8-specific agonist).

As an example, one TLR8-mediated biological activity can include production of tumor necrosis factor (TNF), which may be beneficial for treating certain conditions such as, for example, certain cancers (e.g., melanoma). On the other hand, TLR7-mediated biological activity can include production of interferon-α (IFN-α), which may aggravate certain conditions such as, for example, lupus erythematosus. A particular TLR7/8 agonist may be identified as being well-suited for treating certain cancers such as, for example, melanoma, perhaps because of efficacy and/or the extent of TLR8-mediated biological activity induced by the compound, but also perhaps because of other desirable characteristics such as, for example, low toxicity, being easy to formulate and deliver (formulability), cost, stability (e.g., shelf-life), bio-availability, metabolic half-life, etc. However, if administered to a subject having lupus erythematosus, the TLR7-mediated biological activity (IFN-a production) induced by the compound may aggravate the lupus erythematosus to an extent that may prevent consideration of the TLR7/8 compound as a treatment for cancer in a patient that has been diagnosed with lupus erythematosus.

Practicing the present invention may allow such a subject to enjoy the benefits of treating one condition (e.g., the cancer) with the TLR7/8 compound without aggravating the second condition (e.g., lupus erythematosus) to an intolerable extent. By administering a sufficient amount of an immunomodulatory oligonucleotide with the TLR7/8 agonist, sufficient TLR8-mediated biological activity may be induced by the TLR7/8 compound to provide treatment for the cancer, while the TLR7-mediated biological activity induced by the TLR7/8 compound may be reduced to acceptable levels—in some cases, even fully eliminating the TLR7-mediated biological activity. Thus, in the example above, administering the combination of the TLR7/8 agonist and immunomodulatory oligonucleotide may induce sufficient TNF to treat the cancer and reduce the amount of IFN-α induced by the TLR7/8 agonist sufficiently so that the treatment of the cancer may proceed while limiting—or even eliminating—aggravation of the lupus erythematosus that would otherwise result from administering the TLR7/8 agonist.

In still other cases, the method may be used to decrease the TLR7-mediated biological activity induced by certain IRM compounds that, if not so limited, may be too great for the IRM compound to be clinically useful. For example, a TLR7 agonist may be desirable for development for clinical use for one or more of a number of reasons (e.g., ease or cost of synthesis, toxicity, formulability, etc.), but may be superpotent—i.e., too potent of an inducer of TLR7-mediated biological activity (e.g., IFN-α production) to be clinically useful. In such cases, combining the IRM compound with an immunomodulatory oligonucleotide may reduce the extent to which the TLR7 agonist induces TLR7-mediated biological activity to within the clinically acceptable range. A TLR7 agonist may be used to treat or prevent, for example, a chronic viral infection (e.g., hepatitis C) or a metastatic cancer (e.g., melanoma). Administering the TLR7 agonist can induce an innate immune response that may include IFN-α induction. However, induction of too much IFN-α could cause undesirable side affects (e.g. strong flu-like symptoms, vomiting, etc.). Thus, an immunomodulatory oligonucleotide may be combined with a superpotent TLR7 agonist so that the level of IFN-α induced in a subject by the TLR7 agonist is reduced, thereby tempering the severity of IFN-α-induced side effects to manageable or acceptable levels while maintaining a therapeutic or prophylactic level of IFN-α induction for the condition being treated (e.g., viral infection or cancer).

In still other cases, the method may be used to permit local administration of a TLR7 agonist to generate a strong local therapeutic or prophylactic immune response while limiting the extent to which the TLR7-mediated biological activity induced by the TLR7 agonist causes undesirable systemic side effects. For example, the TLR7 agonist may be administered locally as a prophylactic influenza treatment (e.g., administered intranasally) or a therapeutic treatment for lung cancer (e.g., administered by inhalation), thereby generating a generally localized TLR7-mediated immune response. An immunomodulatory oligonucleotide may be administered in a manner and via a route appropriate to reduce any systemic TLR7-mediated side effects that can result from administration of the TLR7 agonist.

Thus, in another aspect, the invention provides immunomodulatory compositions that are effective for reducing TLR7-mediated biological activity. In some cases, the composition can include an immunomodulatory oligonucleotide in an amount effective to reduce TLR7-mediated biological activity. In other cases, the invention provides an immunomodulatory combination that can include a TLR7 agonist and an immunomodulatory oligonucleotide in an amount effective to reduce TLR7-mediated biological activity induced by the TLR7 agonist. In some cases, the TLR7 agonist also may be an agonist of at least one other TLR (e.g., TLR8—a TLR7/8 agonist), so that the immunomodulatory combination includes an IRM compound that is an agonist of TLR7 and at least one other TLR and an immunomodulatory oligonucleotide in an amount effective to reduce TLR7-mediated biological activity induced by the IRM compound.

In embodiments in which the immunomodulatory combination includes an immunomodulatory oligonucleotide and a TLR7 agonist, the two components may exist in a single formulation. Alternatively, the two components may exist in separate formulations such as, for example, in the example described above in which the TLR7 agonist is administered locally and the immunomodulatory oligonucleotide is administered separately from the TLR7 agonist.

Exemplary TLR7-mediated biological activities that may be modulated while practicing the invention can include, for example, induction of co-stimulatory marker expression, induction of surface marker expression, increased antigen-presenting capability, maturation of plasmacytoid dendritic cells (pDCs), proliferation of B lymphocytes, and induction of certain cytokines. Cytokines induced by a TLR7-mediated biological activity include, for example, IFN-α, IP-10, and MIP.

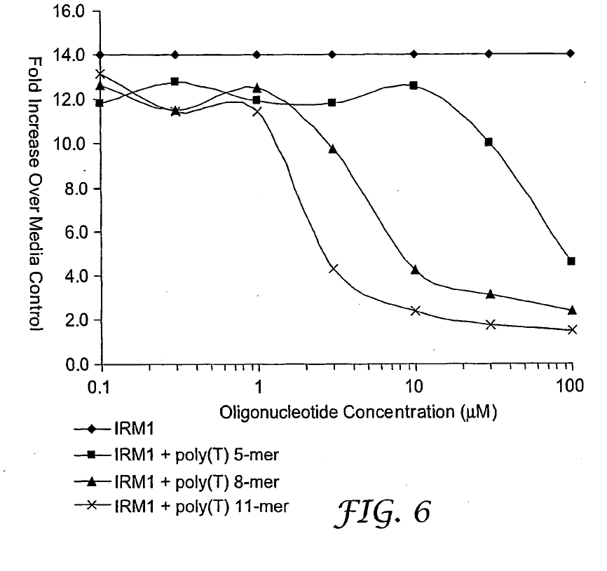

The immunomodulatory oligonucleotide may be any suitable oligonucleotide sequence. Generally, the oligonucleotide can be at least five bases in length such as, for example, at least eight bases in length or at least 11 bases in length (FIG. 6). In some embodiments, a suitable immunomodulatory oligonucleotide may be no more than 14 bases in length such as, for example, no more than 11 bases in length or no more than eight bases in length. Thus, a suitable immunomodulatory oligonucleotide may be, for example, from five to 14 bases in length, from eight to 14 bases in length, from 11 to 14 bases in length, from five to 11 bases in length, etc. In still other embodiments, a suitable immunomodulatory oligonucleotide may be, for example, at least 26 bases in length such as, for example, at least 30 bases in length or at least 45 bases in length.

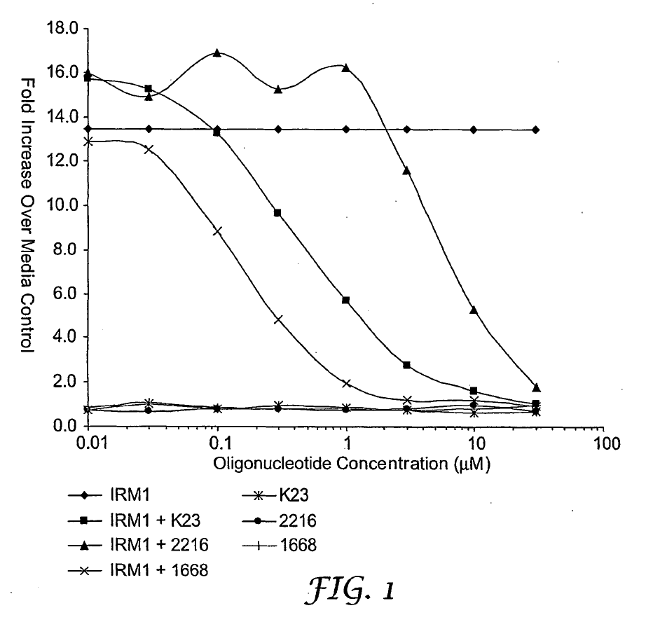

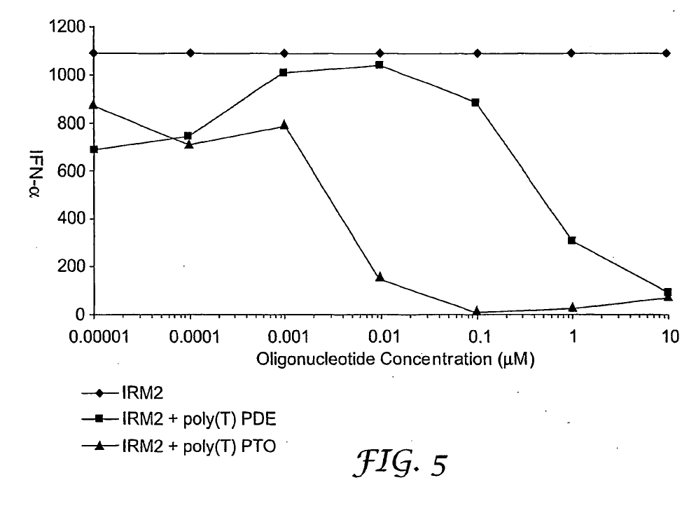

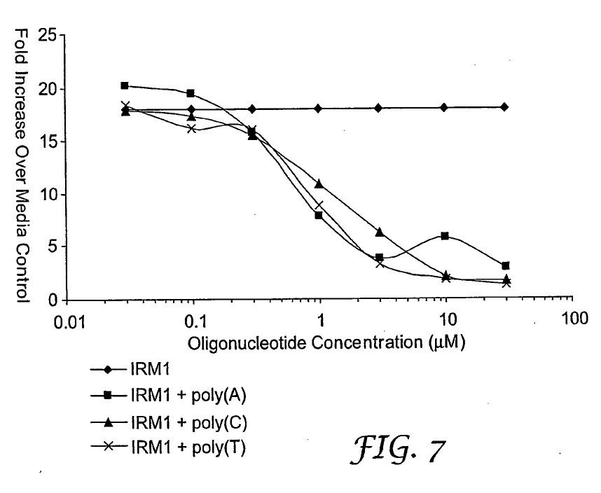

In some embodiments, a suitable immunomodulatory oligonucleotide may contain CpG ODN sequences such as, for example, CpG-A ODN, CpG-B ODN, or CpG-C ODN sequences (FIGS. 1-4). However, other oligonucleotide sequences may be suitable as well. For example, poly(A), poly(C) and poly(T) oligonucleotides have been identified as being capable of limiting TLR7-mediated biological activity (FIG. 5 and FIG. 7).

In some embodiments, the immunomodulatory oligonucleotide can have a stacked secondary structure that may permit the IRM compound to intercalate into the oligonucleotide sequence. Intercalation of the IRM compound into the oligonucleotide may impair the ability of the IRM compound to participate in an agonist-receptor interaction that would otherwise induce TLR7-mediated biological activity.

Certain IRMs are small organic molecules (smIRMs, e.g., molecular weight under about 1000 Daltons, in some cases under about 500 Daltons, as opposed to large biological molecules such as proteins, peptides, and the like) such as those disclosed in, for example, U.S. Pat. Nos. 4,689,338; 4,929,624; 4,988,815; 5,037,986; 5,175,296; 5,238,944; 5,266,575; 5,268,376; 5,346,905; 5,352,784; 5,367,076; 5,389,640; 5,395,937; 5,446,153; 5,482,936; 5,693,811; 5,741,908; 5,756,747; 5,939,090; 6,039,969; 6,083,505; 6,110,929; 6,194,425; 6,245,776; 6,331,539; 6,376,669; 6,451,810; 6,525,064; 6,541,485; 6,545,016; 6,545,017; 6,558,951; 6,573,273; 6,656,938; 6,660,735; 6,660,747; 6,664,260; 6,664,264; 6,664,265; 6,667,312; 6,670,372; 6,677,347; 6,677,348; 6,677,349; 6,683,088; 6,756,382; European Patent 0 394 026; U.S. Patent Publication Nos. 2002/0016332; 2002/0055517; 2002/0110840; 2003/0133913; 2003/0199538; and 2004/0014779; and International Patent Publication Nos. WO 01/74343; WO 02/46749 WO 02/102377; WO 03/020889; WO 03/043572; WO 03/045391; WO 03/103584; and WO 04/058759.

Additional examples of small molecule IRMs include certain purine derivatives (such as those described in U.S. Pat. Nos. 6,376,501, and 6,028,076), certain imidazoquinoline amide derivatives (such as those described in U.S. Pat. No. 6,069,149), certain imidazopyridine derivatives (such as those described in U.S. Pat. No. 6,518,265), certain benzimidazole derivatives (such as those described in U.S. Pat. No. 6,387,938), certain derivatives of a 4-aminopyrimidine fused to a five membered nitrogen containing heterocyclic ring (such as adenine derivatives described in U.S. Pat. Nos. 6,376,501; 6,028,076 and 6,329,381; and in WO 02/08905), and certain 3-β-D-ribofuranosylthiazolo[4,5-d]pyrimidine derivatives (such as those described in U.S. Publication No. 2003/0199461).

Other IRMs include large biological molecules such as oligonucleotide sequences. Some IRM oligonucleotide sequences contain cytosine-guanine dinucleotides (CpG) and are described, for example, in U.S. Pat. Nos. 6,194,388; 6,207,646; 6,239,116; 6,339,068; and 6,406,705. Some CpG-containing oligonucleotides can include synthetic immunomodulatory structural motifs such as those described, for example, in U.S. Pat. Nos. 6,426,334 and 6,476,000. Other IRM nucleotide sequences lack CpG sequences and are described, for example, in International Patent Publication No. WO 00/75304.

Other IRMs include biological molecules such as aminoalkyl glucosaminide phosphates (AGPs) and are described, for example, in U.S. Pat. Nos. 6,113,918; 6,303,347; 6,525,028; and 6,649,172.

Unless otherwise indicated, reference to a compound can include the compound in any pharmaceutically acceptable form, including any isomer (e.g., diastereomer or enantiomer), salt, solvate, polymorph, and the like. In particular, if a compound is optically active, reference to the compound can include each of the compound's enantiomers as well as racemic mixtures of the enantiomers.

In some embodiments of the present invention, the IRM compound may include a 2-aminopyridine fused to a five membered nitrogen-containing heterocyclic ring, or a 4-aminopyrimidine fused to a five membered nitrogen-containing heterocyclic ring.

IRM compounds suitable for use in the invention include compounds having a 2-aminopyridine fused to a five membered nitrogen-containing heterocyclic ring. Such compounds include, for example, imidazoquinoline amines including but not limited to substituted imidazoquinoline amines such as, for example, amide substituted imidazoquinoline amines, sulfonamide substituted imidazoquinoline amines, urea substituted imidazoquinoline amines, aryl ether substituted imidazoquinoline amines, heterocyclic ether substituted imidazoquinoline amines, amido ether substituted imidazoquinoline amines, sulfonamido ether substituted imidazoquinoline amines, urea substituted imidazoquinoline ethers, thioether substituted imidazoquinoline amines, and 6-, 7-, 8-, or 9-aryl or heteroaryl substituted imidazoquinoline amines; tetrahydroimidazoquinoline amines including but not limited to amide substituted tetrahydroimidazoquinoline amines, sulfonamide substituted tetrahydroimidazoquinoline amines, urea substituted tetrahydroimidazoquinoline amines, aryl ether substituted tetrahydroimidazoquinoline amines, heterocyclic ether substituted tetrahydroimidazoquinoline amines, amido ether substituted tetrahydroimidazoquinoline amines, sulfonamido ether substituted tetrahydroimidazoquinoline amines, urea substituted tetrahydroimidazoquinoline ethers, and thioether substituted tetrahydroimidazoquinoline amines; imidazopyridine amines including but not limited to amide substituted imidazopyridine amines, sulfonamide substituted imidazopyridine amines, urea substituted imidazopyridine amines, aryl ether substituted imidazopyridine amines, heterocyclic ether substituted imidazopyridine amines, amido ether substituted imidazopyridine amines, sulfonamido ether substituted imidazopyridine amines, urea substituted imidazopyridine ethers, and thioether substituted imidazopyridine amines; 1,2-bridged imidazoquinoline amines; 6,7-fused cycloalkylimidazopyridine amines; imidazonaphthyridine amines; tetrahydroimidazonaphthyridine amines; oxazoloquinoline amines; thiazoloquinoline amines; oxazolopyridine amines; thiazolopyridine amines; oxazolonaphthyridine amines; thiazolonaphthyridine amines; and 1H-imidazo dimers fused to pyridine amines, quinoline amines, tetrahydroquinoline amines, naphthyridine amines, or tetrahydronaphthyridine amines.

In certain embodiments, the IRM compound may be an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine.

In certain other embodiments, the IRM compound may be a substituted imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine.

As used herein, a substituted imidazoquinoline amine refers to an amide substituted imidazoquinoline amine, a sulfonamide substituted imidazoquinoline amine, a urea substituted imidazoquinoline amine, an aryl ether substituted imidazoquinoline amine, a heterocyclic ether substituted imidazoquinoline amine, an amido ether substituted imidazoquinoline amine, a sulfonamido ether substituted imidazoquinoline amine, a urea substituted imidazoquinoline ether, a thioether substituted imidazoquinoline amines, or a 6-, 7-, 8-, or 9-aryl or heteroaryl substituted imidazoquinoline amine. As used herein, substituted imidazoquinoline amines specifically and expressly exclude 1-(2-methylpropyl)-1H-imidazo[4,5-c]quinolin-4-amine and 4-amino-α,α-dimethyl-2-ethoxymethyl-1H-imidazo[4,5-c]quinolin-1-ethanol.

In certain embodiments, the IRM compound may be a tetrahydroimidazoquinoline amine such as, for example, 4-amino-2-(ethoxymethyl)-α,α-dimethyl-6,7,8,9-tetrahydro-1H-imidazo[4,5-c]quinoline-1-ethanol.

In other embodiments, the IRM compound may be a sulfonamide substituted imidazoquinoline amine such as, for example, N-[4-(4-amino-2-ethyl-1H-imidazo[4,5-c]quinolin-1-yl)butyl]methanesulfonamide, N-[4-(4-amino-2-propyl-1H-imidazo[4,5-c]quinolin-1-yl)butyl]methanesulfonamide, or N-[4-(4-amino-2-butyl-1H-imidazo[4,5-c]quinolin-1-yl)butyl]methanesulfonamide.

In other embodiments, the IRM compound may be a naphthyridine amine such as, for example, 2-methyl-1-(2-methylpropyl)-1H-imidazo[4,5-c][1,5]naphthyridin-4-amine or 1-(2-methylpropyl)-1H-imidazo[4,5-c][1,5]naphthyridin-4-amine.

In still other embodiments, the IRM compound may be a urea substituted tetrahydroimidazoquinoline amine such as, for example, N-[4-(4-amino-2-methyl-6,7,8,9,-tetrahydro-1H-imidazo[4,5-c]quinolin-1-yl)butyl]morpholine-4-carboxamide.

Suitable IRM compounds also may include the purine derivatives, imidazoquinoline amide derivatives, benzimidazole derivatives, adenine derivatives, aminoalkyl glucosaminide phosphates, and oligonucleotide sequences described above.

An immunomodulatory composition may be provided in a formulation that includes an immunomodulatory oligonucleotide. In other cases, an immunomodulatory combination may include an immunomodulatory oligonucleotide and an IRM compound. Alternatively, an immunomodulatory combination may include a plurality of formulations in which the IRM compound and the immunomodulatory oligonucleotide may be provided in the same formulation or in different formulations. Formulations suitable for use in connection with therapeutic compositions and combinations of the invention are described in detail below.

An immunomodulatory composition or combination may be provided in any formulation or combination of formulations suitable for administration to a subject. Suitable types of formulations are described, for example, in U.S. Pat. No. 5,736,553; U.S. Pat. No. 5,238,944; U.S. Pat. No. 5,939,090; U.S. Pat. No. 6,365,166; U.S. Pat. No. 6,245,776; U.S. Pat. No. 6,486,186; European Patent No. EP 0 394 026; and International Patent Publication No. WO 03/045391. A formulation may be provided in any suitable form including, but not limited to, a solution, a suspension, an emulsion, or any form of mixture. A formulation may include any pharmaceutically acceptable excipient, carrier, or vehicle. For example, a formulation may be delivered in a conventional dosage form such as, for example, a cream, an ointment, an aerosol formulation, a non-aerosol spray, a gel, a lotion, a tablet, an elixir, and the like. A formulation may further include one or more additives including but not limited to adjuvants, skin penetration enhancers, colorants, flavorings, fragrances, moisturizers, thickeners, and the like.

A formulation may be administered in any suitable manner such as, for example, non-parenterally or parenterally. As used herein, non-parenterally refers to administration through the digestive tract, including by oral ingestion. Parenterally refers to administration other than through the digestive tract such as, for example, intravenously, intramuscularly, transdermally, subcutaneously, transmucosally (e.g., by inhalation), or topically.

The composition of a formulation suitable for practicing the invention may vary according to factors known in the art including but not limited to the physical and chemical nature of the immunomodulatory oligonucleotide, the nature of the carrier, the intended dosing regimen, the state of the subject's immune system (e.g., suppressed, compromised, stimulated), the method of administering the immunomodulatory oligonucleotide, the nature and potency of any TLR7 agonist administered with the immunomodulatory oligonucleotide (if any), and the species to which the formulation is being administered. Accordingly, it is not practical to set forth generally the composition of a formulation effective for all possible applications. Those of ordinary skill in the art, however, can readily determine an appropriate formulation with due consideration of such factors.

In some embodiments, the methods of the present invention include administering immunomodulatory oligonucleotide to a subject in a formulation of, for example, from about 0.0001% to about 10% (unless otherwise indicated, all percentages provided herein are weight/weight with respect to the total formulation) to the subject, although in some embodiments the immunomodulatory oligonucleotide may be administered using a formulation that provides immunomodulatory oligonucleotide in a concentration outside of this range. For example, a formulation may include from about 0.01% to about 1% immunomodulatory oligonucleotide.

In some embodiments, the methods of the present invention further include administering IRM to a subject in a formulation of, for example, from about 0.0001% to about 10% to the subject, although in some embodiments the IRM compound may be administered using a formulation that provides IRM compound in a concentration outside of this range. In certain embodiments, the method includes administering to a subject a formulation that includes from about 0.01% to about 5% IRM compound, for example, a formulation that includes from about 0.1% to about 0.5% IRM compound.

An amount of an immunomodulatory oligonucleotide effective for reducing TLR7-mediated biological activity of immune cells is an amount sufficient to reduce at least one TLR7-mediated biological activity. The precise amount of immunomodulatory oligonucleotide required to be effective may vary according to factors known in the art such as, for example, the physical and chemical nature of the immunomodulatory oligonucleotide, the nature of the carrier, the intended dosing regimen, the state of the subject's immune system (e.g., suppressed, compromised, stimulated), the method of administering the immunomodulatory oligonucleotide, the potency of any TLR7 agonist being administered with the immunomodulatory oligonucleotide (if any), and the species to which the formulation is being administered. Accordingly, it is not practical to set forth generally the amount that constitutes an amount of immunomodulatory oligonucleotide effective for all possible applications. Those of ordinary skill in the art, however, can readily determine the appropriate amount with due consideration of such factors. In some embodiments, the methods of the present invention include administering sufficient immunomodulatory oligonucleotide to provide a dose of, for example, from about 100 ng/kg to about 50 mg/kg to the subject, although in some embodiments the methods may be performed by administering immunomodulatory oligonucleotide in a dose outside this range. In some of these embodiments, the method includes administering sufficient immunomodulatory oligonucleotide to provide a dose of from about 10 μg/kg to about 5 mg/kg to the subject, for example, a dose of from about 100 μg/kg to about 1 mg/kg.

The dosing regimen may depend at least in part on many factors known in the art including but not limited to the physical and chemical nature of the immunomodulatory oligonucleotide, the nature of the carrier, the amount of immunomodulatory oligonucleotide being administered, the state of the subject's immune system (e.g., suppressed, compromised, stimulated), the method of administering the immunomodulatory oligonucleotide, the desired result, and the potency of any TLR7 agonist being administered with the immunomodulatory oligonucleotide (if any), and the species to which the formulation is being administered. Accordingly it is not practical to set forth generally the dosing regimen effective for all possible applications. Those of ordinary skill in the art, however, can readily determine an appropriate dosing regimen with due consideration of such factors.

In some embodiments, the immunomodulatory oligonucleotide may be administered on an “as needed” basis if being used, for example, to reduce the TLR7-mediated biological activity induced by administering a dose of a TLR7 agonist that is greater than necessary. In some cases, the immunomodulatory oligonucleotide may be administered only once. In other embodiments, the immunomodulatory oligonucleotide may be administered with respect to the administration of a TLR7 agonist. In such cases, the immunomodulatory oligonucleotide may be administered in an immunomodulatory oligonucleotide:IRM compound ratio of from about 1:1000 to about 30:1, although in some embodiments the methods of the present invention may be performed by administering the immunomodulatory oligonucleotide in an immunomodulatory oligonucleotide:IRM compound ratio outside this range. In certain embodiments, the immunomodulatory oligonucleotide may be administered in an immunomodulatory oligonucleotide:IRM compound ratio of at least 1:500, 1:100, 1:30, 1:10, 1:3 or 1:1 In certain embodiments, the immunomodulatory oligonucleotide may be administered in an immunomodulatory oligonucleotide:IRM compound ratio of no more than 30:1, 10:1, 5:1, 3:1, 1:1, 1:3, or 1:10. In one particular embodiment, the immunomodulatory oligonucleotide may be administered in an immunomodulatory oligonucleotide:IRM compound ratio of about 1:1.

Conditions that may be treated by practicing the invention include, but are not limited to:

(a) viral diseases such as, for example, diseases resulting from infection by an adenovirus, a herpesvirus (e.g., HSV-I, HSV-II, CMV, or VZV), a poxvirus (e.g., an orthopoxvirus such as variola or vaccinia, or molluscum contagiosum), a picornavirus (e.g., rhinovirus or enterovirus), an orthomyxovirus (e.g., influenzavirus), a paramyxovirus (e.g., parainfluenzavirus, mumps virus, measles virus, and respiratory syncytial virus (RSV)), a coronavirus (e.g., SARS), a papovavirus (e.g., papillomaviruses, such as those that cause genital warts, common warts, or plantar warts), a hepadnavirus (e.g., hepatitis B virus), a flavivirus (e.g., hepatitis C virus or Dengue virus), or a retrovirus (e.g., a lentivirus such as HIV);

(b) bacterial diseases such as, for example, diseases resulting from infection by bacteria of, for example, the genus Escherichia, Enterobacter, Salmonella, Staphylococcus, Shigella, Listeria, Aerobacter, Helicobacter, Klebsiella, Proteus, Pseudomonas, Streptococcus, Chlamydia, Mycoplasma, Pneumococcus, Neisseria, Clostridium, Bacillus, Corynebacterium, Mycobacterium, Carnpylobacter, Vibrio, Serratia, Providencia, Chromobacterium, Brucella, Yersinia, Haemophilus, or Bordetella;

(c) other infectious diseases, such chlamydia, fungal diseases including but not limited to candidiasis, aspergillosis, histoplasmosis, cryptococcal meningitis, or parasitic diseases including but not limited to malaria, pneumocystis carnii pneumonia, leishmaniasis, cryptosporidiosis, toxoplasmosis, and trypanosome infection; and

(d) neoplastic diseases, such as intraepithelial neoplasias, cervical dysplasia, actinic keratosis, basal cell carcinoma, squamous cell carcinoma, Kaposi's sarcoma, melanoma, renal cell carcinoma, leukemias including but not limited to myelogeous leukemia, chronic lymphocytic leukemia, multiple myeloma, non-Hodgkin's lymphoma, cutaneous T-cell lymphoma, B-cell lymphoma, and hairy cell leukemia, and other cancers;

(e) T_{H}2-mediated, atopic diseases, such as atopic dermatitis or eczema, eosinophilia, asthma, allergy, allergic rhinitis, and Ommen's syndrome;

(f) certain autoimmune diseases such as systemic lupus erythematosus, essential thrombocythaemia, multiple sclerosis, discoid lupus, alopecia greata; and

(g) diseases associated with wound repair such as, for example, inhibition of keloid formation and other types of scarring (e.g., enhancing wound healing, including chronic wounds).

Additionally, an immunomodulatory oligonucleotide (or immunomodulatory combination that includes and IRM compound and an immunomodulatory oligonucleotide) may be useful as a vaccine adjuvant for use in conjunction with any material that raises either humoral and/or cell mediated immune response, such as, for example, live viral, bacterial, or parasitic immunogens; inactivated viral, tumor-derived, protozoal, organism-derived, fungal, or bacterial immunogens, toxoids, toxins; self-antigens; polysaccharides; proteins; glycoproteins; peptides; cellular vaccines; DNA vaccines; autologous vaccines; recombinant proteins; glycoproteins; peptides; and the like, for use in connection with, for example, BCG, cholera, plague, typhoid, hepatitis A, hepatitis B, hepatitis C, influenza A, influenza B, parainfluenza, polio, rabies, measles, mumps, rubella, yellow fever, tetanus, diphtheria, hemophilus influenza b, tuberculosis, meningococcal and pneumococcal vaccines, adenovirus, HIV, chicken pox, cytomegalovirus, dengue, feline leukemia, fowl plague, HSV-1 and HSV-2, hog cholera, Japanese encephalitis, respiratory syncytial virus, rotavirus, papilloma virus, yellow fever, and Alzheimer's Disease.

The methods of the present invention may be performed on any suitable subject. Suitable subjects include but are not limited to animals such as but not limited to humans; non-human primates, rodents, dogs, cats, horses, pigs, sheep, goats, or cows.

### EXAMPLES

The following examples have been selected merely to further illustrate features, advantages, and other details of the invention. It is to be expressly understood, however, that while the examples serve this purpose, the particular materials and amounts used as well as other conditions and details are not to be construed in a matter that would unduly limit the scope of this invention.

The IRM compounds used in the examples are shown in Table 1. The immunomodulatory oligonucleotides used in the examples are shown in Table 2.

TABLE 1

| Compound | Chemical Name | Reference |
|---|---|---|
| IRM1 | 4-amino-2-(ethoxymethyl)-α,α-dimethyl- | U.S. Pat. No. |
| | 6,7,8,9-tetrahydro-1H-imidazo[4,5- | 5,352,784 |
| | c]quinoline-1-ethanol | Example 91 |
| IRM2 | N-[4-(4-amino-2-ethyl-1H-imidazo[4,5- | U.S. Pat. No. |
| | c]quinolin-1-yl)butyl]methanesulfonamide | 6,677,349 |
| | | Example 236 |

TABLE 2

| | | |
|---|---|---|
| SEQ ID | CpG/type | Sequence* |

| | | |
|---|---|---|
| SEQ ID NO:1 | K23/B | 5′-TCGAGCGTTGTC-3′ |
| SEQ ID NO:2 | 2216/A | 5′-GGgggacgatcgtcGGGGG |
| | | g-3′ |
| SEQ ID NO:3 | 1668/Murine | 5′-TCCATGACGTTCCTGATGC |
| | | T-3′ |
| SEQ ID NO:4 | 2006/B | 5′-TCGTCGTTTTGTCGTTTTGTCG |
| | | TT-3′ |
| SEQ ID NO:5 | M352/C | 5′-TCGTCGAACGTTCGAGATGA |
| | | T-3′ |
| SEQ ID NO:6 | | 5′-TTTTTTTTTTTTTTTTTTTT |
| | | T-3′ |
| SEQ ID NO:7 | | 5′-tttttttttttttttttttt |
| | | t-3′ |
| SEQ ID NO:8 | | 5′-TTTTT-3′ |
| SEQ ID NO:9 | | 5′-TTTTTTTT-3′ |
| SEQ ID NO:10 | | 5′-TTTTTTTTTTT-3′ |
| SEQ ID NO:11 | | 5′-TTTTTTTTTTTTTTTTTT-3′ |
| SEQ ID NO:12 | | 5′-AAAAAAAAAAAAAAAAAA-3′ |
| SEQ ID NO:13 | | 5′-CCCCCCCCCCCCCCCCCC-3′ |

*Upper case letters indicate a phosporothioate linkage 3′ of the base;
lower case letters indicate a phosphocliester linkage 3′ of the base.

SEQ ID NO: 1 is reported in Gürsel et al., J. Leukoc. Biol. (2002), vol. 71, pp. 813-820. SEQ ID NO:2, SEQ ID NO:4, and SEQ ID NO:5 are reported in Hartmenn et al., Eur. J. Immunol. (2003), vol. 33, pp. 1633-1641. SEQ ID NO:3 is reported in Zhu et al., J. Leukoc. Biol. (2002), vol. 72, pp. 1154-1163.

### Example 1

Human TLR7 and NFκβ were transfected into human epithelial kidney 293 (HEK293, American Type Culture Collection, Manassas, Va., ATCC No. CRL-1573) cells as described in U.S. Patent Publication Nos. U82004/0014779 and US2004/0171086. The selected transfected cells were counted and resuspended to a concentration of 5×10⁵ cell per mL in culture media.

Cultured media was prepared from complete DMEM media (Biosource International Inc., Camarillo, Calif.), without phenol red. Fetal bovine serum (Biosource International Inc.) was added to a final concentration of 10% (vol/vol.), sodium pyruvate (Biosource International Inc.) was added to 1 mM; L-glutamine (Biosource International Inc.) was added to 2 mM; penicillin (Biosource International Inc.) was added to 100 U/mL; streptomycin (Biosource International Inc.) was added to 100 μg/mL.

100 μL aliquots of cells were placed in the wells of a white-walled, white-bottomed 96-well plate (Corning, Inc. Corning, N.Y.). Cell aliquots were treated by adding CpG ODN K23 (SEQ ID NO:1), 2216 (SEQ ID NO:2), 1668 (SEQ ID NO:3), 2006 (SEQ ID NO:4) or M352 (SEQ ID NO:5) (Invitrogen Corp., Carlsbad, Calif.) at a concentration of 0.01 μM, 0.03 μM, 0.1 μM, 0.3 μM, 1.0 μM, 3.0 μM, 10 μM, or 30 μM to the culture with or without either 3 μM IRM1 or 10 μM IRM2. As a positive control, some cell aliquots were incubated with either 3 μM IRM1 or 10 μM IRM2. As a negative control, some cell aliquots were incubated without a stimulus (media control). In all cases, the cells were incubated overnight at 37° C. with 5% CO₂ and 98% humidity.

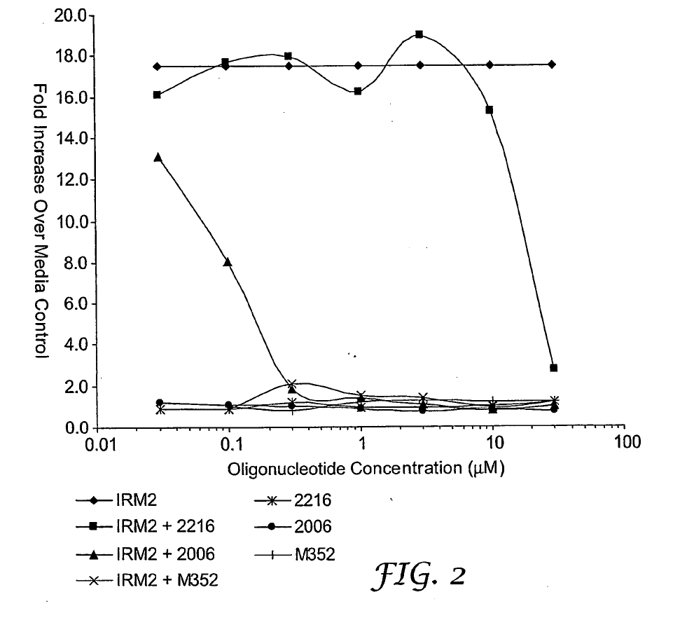

After the cells incubated overnight, 100 μL volume of reconstituted LucLight Plus (Packard Instruments, Meriden, Conn.) was added to each aliquot of cells. Each well of the plate was read on a L-max luminometer (Molecular Devices, Sunnyvale, Calif.). The data is expressed as fold increase of luciferase induction in cell aliquots incubated with the indicated stimulant compared to the negative control. Results are shown in FIG. 1 and FIG. 2.

### Example 2

Peripheral blood mononeuclear cells (PBMCs) were enriched from human peripheral blood by HISTOPAQUE-1077 (Sigma-Aldrich Co., St. Louis, Mo.) density gradient centrifugation. PBMCs were counted and resuspended in complete RPMI 1640 with 25 mM HEPES (Biosource International Inc.) media. Fetal bovine serum (Biosource International Inc.) was added to a final concentration of 10% (vol/vol.), L-glutamine (Biosource International Inc.) was added to 2 mM; penicillin (Biosource International Inc.) was added to 100 U/mL; streptomycin (Biosource International Inc.) was added to 100 μg/mL.

5×10⁵ cell per well in 200 μL placed in flat-bottom 96-well plate (Becton Dickenson Labware, Franklin Lakes, N.J.). Cell aliquots were treated by adding 1 μM of IRM2 alone (positive control) or with CpG ODN K23 (SEQ ID NO:1) or 2006 (SEQ ID NO:4) (Invitrogen Corp.) at a concentration of 0.1 μM, 0.3 μM, 1.0 μM, 3.0 μM, 10 μM, or 30 μM. In all cases, the cells were incubated overnight at 37° C. with 5% CO₂ and 98% humidity.

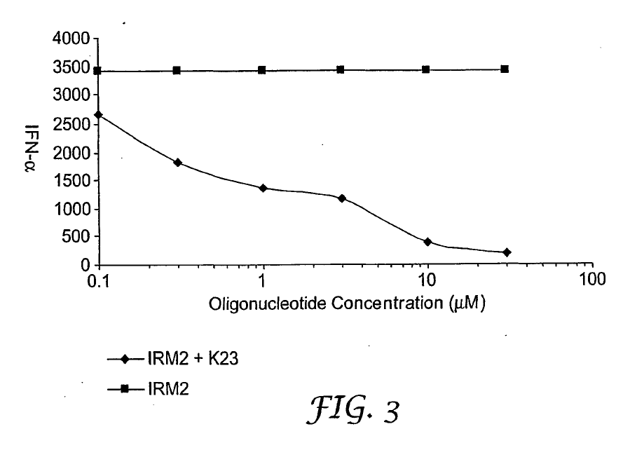

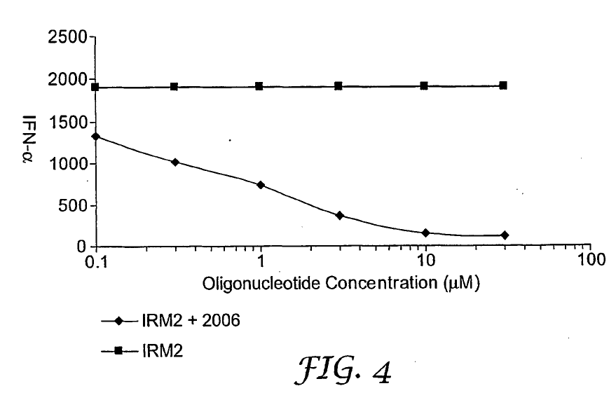

Culture supernatants were analyzed for IFN-α (pg/mL) production using a human-specific IFN-α ELISA (PBL Biomedical Lab., Piscataway, N.J.). Results are shown in FIG. 3 and FIG. 4.

### Example 3

PBMCs were prepared as described in Example 2. Cell aliquots were treated by adding 1 μM of IRM2 alone (positive control) or with a 20-mer thymine poly(T) oligonucleotide sequence containing either a phosphodiester (PDE, SEQ ID NO:7) or phosphorothioate (PTO, SEQ ID NO:6) backbone (Invitrogen Corp.) at a concentration of 0.00001 μM, 0.0001 μM, 0.001 μM, 0.01 μM, 0.1 μM, 1.0 μM, or 10 μM. Culture supernatants were analyzed for IFN-α production using a human-specific IFN-α (pg/mL) ELISA (PBL Biomedical Lab.). Results shown in FIG. 5 represent the average of two experiments.

### Example 4

HEK293 cells expressing human TLR7 were prepared as described in Example 1. Cell aliquots were treated with 3 μM of IRM1 alone (positive control) or with a 5-mer (SEQ ID NO:8), 8-mer (SEQ ID NO:9), or 11-mer (SEQ ID NO:10) poly(T) oligonucleotide sequence (Invitrogen Corp.) at a concentration of 0.1 μM, 0.3 μM, 1.0 μM, 3.0 μM, 10 μM, 30 μM, or 100 μM. As a negative control, some cell aliquots were incubated without a stimulus (media control).

After the cells incubated overnight, the cells were analyzed as described in Example 1. The data is expressed as fold increase of luciferase induction in cell aliquots incubated with the indicated stimulant compared to the negative control. Results are shown in FIG. 6.

### Example 5

HEK293 cells expressing human TLR7 were prepared as described in Example 1. Cell aliquots were treated with 3 μM of IRM1 alone (positive control) or with an 18-mer poly(T) oligonucleotide (SEQ ID NO: 11), poly(A) oligonucleotide (SEQ ID NO: 12), or poly(C) oligonucleotide (SEQ ID NO:13) (Invitrogen Corp.) at a concentration of 0.03 μM, 0.1 μM, 0.3 μM, 1.0 μM, 3.0 μM, 10 μM, or 30 μM. As a negative control, some cell aliquots were incubated without a stimulus (media control).

After the cells incubated overnight, the cells were analyzed as described in Example 1. The data is expressed as fold increase of luciferase induction in cell aliquots incubated with the indicated stimulant compared to the negative control. Results are shown in FIG. 7.

The complete disclosures of the patents, patent documents and publications cited herein are incorporated by reference in their entirety as if each were individually incorporated. In case of conflict, the present specification, including definitions, shall control.

Various modifications and alterations to this invention will become apparent to those skilled in the art without departing from the scope and spirit of this invention. Illustrative embodiments and examples are provided as examples only and are not intended to limit the scope of the present invention. The scope of the invention is limited only by the claims set forth as follows.

1-16. (canceled)

17. An immunomodulatory combination that comprises:
a TLR7 agonist, wherein the TLR7 agonist is an imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine; and
an immunomodulatory oligonucleotide in an amount effective to reduce at least one TLR7-mediated biological activity induced by the TLR7 agonist.

18. The immunomodulatory combination of claim 17 wherein the immunomodulatory oligonucleotide comprises a CpG oligodinucleotide.

19-23. (canceled)

24. The immunomodulatory combination of claim 17 wherein the TLR7 agonist comprises a TLR7/8 agonist.

25. The immunomodulatory combination of claim 17 wherein the immunomodulatory oligonucleotide comprises from about five bases to 14 bases.

26-27. (canceled)

28. The immunomodulatory combination of claim 17 wherein the immunomodulatory oligonucleotide comprises at least 26 bases.

29. The immunomodulatory combination of claim 17 wherein the immunomodulatory oligonucleotide comprises a poly(T) oligonucleotide.

30. The immunomodulatory combination of claim 17 wherein the immunomodulatory oligonucleotide comprises a poly(A) or poly(C) oligonucleotide.

31. A method of selectively inhibiting TLR7-mediated biological activity of an IRM compound that is an agonist of TLR7 and at least one other TLR agonist, the method comprising:
combining the IRM compound with an immunomodulatory oligonucleotide in an amount effective to reduce TLR7-mediated biological activity induced by the IRM compound, wherein the IRM compound is an imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine; and
contacting the combination of IRM compound and immunomodulatory oligonucleotide with immune cells capable of generating a TLR7-mediated biological response.

32. The method of claim 31 wherein combining the IRM compound with the immunomodulatory oligonucleotide permits formation of an IRM-immunomodulatory oligonucleotide complex.

33. The method of claim 32 wherein the immunomodulatory oligonucleotide comprises a CpG oligodinucleotide.

34-40. (canceled)

41. The method of claim 31 wherein the IRM compound comprises a TLR7/8 agonist.

42-43. (canceled)
